(19) United States
(12) Patent Application Publication
(10) Pub. No.: US 2026/0216140 A1
(43) Pub. Date: Jul. 30, 2026

(54) USE OF ERGOTHIONEINE IN PREPARATION OF PRODUCT FOR PREVENTING OR INHIBITING CELL DAMAGE CAUSED BY ACETALDEHYDE

(71) Applicant: BLOOMAGE BIOTECHNOLOGY CORPORATION LIMITED, Shandong (CN)

(72) Inventors: Liyun DAI, Shandong (CN); Liangliang WEN, Shandong (CN); Yue WU, Shandong (CN); Songyan ZOU, Shandong (CN); Wenjie QU, Shandong (CN)

(73) Assignee: BLOOMAGE BIOTECHNOLOGY CORPORATION LIMITED, Shandong (CN)

(21) Appl. No.: 19/144,180

(22) PCT Filed: Dec. 28, 2023
(86) PCT No.: PCT/CN2023/142561

§ 371 Date: Jun. 27, 2025

(30) Foreign Application Priority Data
Dec. 28, 2022 (CN) 202211694764.6

(51) Int. Cl. A61K 31/4172; A61P 39/02

(52) U.S. Cl. CPC A61K 31/4172; A61P 39/02

(57) ABSTRACT

The present application discloses a use of l-ergothioneine in the preparation of a product for preventing or inhibiting cell damage caused by acetaldehyde. Said l-ergothioneine can improve the survival rate of cells treated with acetaldehyde.

### TECHNICAL FIELD

The present application relates to the technical field of care for cell damage, and in particular to use of ergothioneine in the preparation of a product for preventing or inhibiting cell damage caused by acetaldehyde.

### BACKGROUND ART

In daily life, various substances such as tobacco, alcohol and the relating metabolites may cause damage to cells. For example, after daily diet or drinking alcohol, microorganisms in the oral cavity will produce large amounts of acetaldehyde by alcohol metabolism independently of liver metabolism. At the same time, most alcohol will produce acetaldehyde after being metabolized by the liver, and the acetaldehyde will enter the blood and flow to tissues throughout the body, including the oral cavity. In addition, smoking is also a major source of acetaldehyde in the oral cavity. Cigarettes produce a large amount of acetaldehyde during the burning process. However, due to the lack of ALDH in the microorganisms in the oral cavity and the human body, acetaldehyde cannot be further metabolized into harmless acetic acid in time, so that a large amount of acetaldehyde is accumulated in the special environment of oral cavity, thereby causing serious damage to oral cavity.

As early as 2012, the World Health Organization (WTO) listed acetaldehyde as a Class 1 carcinogen. Epidemiological results show that there is a clear association between drinking alcohol and an increased risk of various cancers in the human body. Nearly 800,000 cancer cases worldwide are caused by alcohol each year. For example, 41% of oral cancers are related to alcohol-derived acetaldehyde.

Acetaldehyde-induced damage is different from conventional wound injury. Common wound injuries mainly involve inflammation induced by cell rupture and invasion of external microorganisms. At the same time, the extracellular matrix supporting the cells will also be damaged. Therefore, the functions of the ingredients conventionally used for treating wound injuries mainly focus on hemostasis, anti-inflammation, and proliferation promotion, etc. Studies have found that acetaldehyde-induced damage mainly involves two aspects: oxidative damage and carcinogenicity. Acetaldehyde has obvious cytotoxicity. It can induce the body to produce a large number of free radicals, thereby causing oxidative damage to macromolecules such as proteins, lipids, and DNA in cells. In addition, acetaldehyde can covalently bind to DNA to form adducts, thereby causing cross-linking and breakage of the DNA double strands, and leading to irreversible cell carcinogenesis. In view of the various hazards of acetaldehyde, how to reduce the damage of acetaldehyde to cell tissues is of great significance.

### SUMMARY

In view of the great harm of acetaldehyde to cells, the present application finds that ergothioneine has the effect of alleviating the cell damage caused by acetaldehyde, thereby completing the present application.

The particular technical solutions of this application are as follows:
1. Use of ergothioneine in the preparation of a product for preventing or inhibiting cell damage caused by acetaldehyde.
2. The use according to item 1, wherein the ergothioneine is used to prevent or inhibit oxidative damage caused by acetaldehyde or to prevent or inhibit cancer caused by acetaldehyde.
3. The use according to item 1 or 2, wherein the site of cell damage caused by acetaldehyde comprises oral cavity, nasal cavity or gastrointestinal tract.
4. The use according to any one of items 1-3, wherein the product comprises an oral preparation or an external preparation.
5. A method for preventing or inhibiting cell damage caused by acetaldehyde, wherein it comprises administering ergothioneine to a site of cell damage caused by acetaldehyde.
6. The method according to item 5, wherein it comprises administering the ergothioneine to the site before or after the site is contacted with acetaldehyde.
7. The method according to item 5 or 6, wherein the site comprises oral cavity, nasal cavity or gastrointestinal tract.
8. The method according to any one of items 5-7, wherein the ergothioneine is used to prevent or inhibit oxidative damage caused by acetaldehyde or to prevent or inhibit cancer caused by acetaldehyde.
9. Use of ergothioneine in the prevention or inhibition of cell damage caused by acetaldehyde.
10. The use according to item 9, wherein the ergothioneine is used to prevent or inhibit oxidative damage caused by acetaldehyde or to prevent or inhibit cancer caused by acetaldehyde.
11. The use according to item 9 or 10, wherein the site of cell damage caused by acetaldehyde comprises oral cavity, nasal cavity or gastrointestinal tract.

### Effects of the Application

The present application applies ergothioneine or a product comprising ergothioneine to the locations of cell damage caused by acetaldehyde, thereby greatly reducing the damage of acetaldehyde to cells, and providing a new theoretical foundation and basis for development of a product for preventing or inhibiting cell damage caused by acetaldehyde.

### BRIEF DESCRIPTION OF THE DRAWINGS

The accompanying drawings are used for better understanding the present application, and do not constitute an improper limitation on the present application, in which:

FIG. 1 shows the relative cell activity of Comparative Example 1.

### DETAILED DESCRIPTION

The present application is described in detail below in conjunction with the described embodiments. Although particular examples of the present application are shown, it should be understood that the present application may be implemented in various forms and should not be limited to the examples set forth herein. On the contrary, these examples are provided to enable a more thorough understanding of the present application and to fully convey the scope of the present application to those skilled in the art.

It should be noted that certain terms are used in the specification and claims to refer to specific components. Those skilled in the art should understand that they may use different terms to refer to the same component. This specification and claims do not use differences in nouns as a way to distinguish components, but rather use differences in functions of components as the criterion for distinction. As mentioned throughout the specification and claims, “comprising” or “including” are open-ended terms, and should be interpreted as “including but not limited to”. The following description are preferred embodiments of the present application, however, the description is for the purpose of the general principles of the specification, and is not intended to limit the scope of the present application. The scope of protection of this application shall be determined by the appended claims.

The present application provides use of ergothioneine in the preparation of a product for preventing or inhibiting cell damage caused by acetaldehyde.

In some embodiments, the ergothioneine is used to prevent or inhibit oxidative damage caused by acetaldehyde or to prevent or inhibit cancer caused by acetaldehyde.

In some embodiments, the site of cell damage caused by acetaldehyde comprises oral cavity, nasal cavity or gastrointestinal tract, for example, comprising oral cell damage, nasal cell damage, respiratory tract cell damage, gastric mucosal cell damage, digestive tract cell damage and other cell damage in the body that may be caused by acetaldehyde, preferably oral cell damage.

The ergothioneine refers to mercaptohistidine betaine, whose chemical name is 2-mercaptohistidine trimethyl internal salt, which is a rare thioimidazole amino acid and also a natural antioxidant. Ergothioneine may be transported into the mitochondria through the transporter OCTN-1 in the cells, directly scavenging reactive oxygen free radicals, and exerting antioxidant and cell-protective effects. In addition, it has multiple physiological functions such as maintaining DNA biosynthesis, normal cell growth, and cellular immunity. At present, ergothioneine is widely used in cosmetics due to its good anti-inflammatory, anti-aging and radiation protection effects, etc.

The inventors of the present application have creatively discovered through a large number of experimental studies that utilizing ergothioneine to treat cells treated with acetaldehyde can improve the activity of the cells, indicating that the ergothioneine can slow down the damage of acetaldehyde to cells, and may be used in oral care, which is of great significance for developing and preparing a product that inhibits cell damage caused by acetaldehyde.

In some embodiments, the product comprises oral preparations and external preparations. The present application does not limit the specific preparation type, and those skilled in the art can choose from the existing technology according to their needs. For example, the oral preparation may be a powder, granule, capsule, liquid preparation, suspension, etc., and the external preparation may be a patch, spray, cream, liquid smear preparation, etc.

In some embodiments, the product comprises an oral care product.

In some embodiments, ergothioneine is used in the preparation of a product for preventing or inhibiting cell damage caused by acetaldehyde. In some embodiments, the ergothioneine is used to prevent or inhibit oxidative damage caused by acetaldehyde or to prevent or inhibit cancer caused by acetaldehyde. In some embodiments, the site of cell damage caused by acetaldehyde comprises oral cavity, nasal cavity or gastrointestinal tract. In some embodiments, the product comprises an oral preparation or an external preparation.

The present application provides a product for preventing or inhibiting cell damage caused by acetaldehyde, which comprises 0.01-220 μM ergothioneine.

In some embodiments, the concentration of ergothioneine in the product is 0.01-12.5 μM, preferably 0.1-220 μM.

For example, the concentration of the ergothioneine in the product may be 0.01 μM, 0.05 μM, 0.1 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 20 μM, 50 μM, 100 μM, 150 μM, 200 μM, or 220 μM, etc.

In the present application, when the product is directly used in cell experiments, the concentration of ergothioneine in the product is 0.01-12.5 μM, preferably 0.1-12.5 μM. When the product is used as an oral or external preparation, the concentration of ergothioneine may change, but the concentration of ergothioneine in the product generally does not exceed 220 μM.

In some embodiments, the product comprises an oral preparation or an external preparation. This application does not limit the specific type of preparations, and those skilled in the art can choose from the existing technology according to their needs. For example, the oral preparations may be a powder, granule, capsule, liquid preparation, suspension, etc., and the external preparations may be a patch, spray, cream, liquid smear preparation, etc.

In some embodiments, the product comprises a mouthwash, toothpaste, powder, lozenge, or oral film. The present application does not impose any limitation on the preparation methods of mouthwash, toothpaste, powder, lozenge or oral film, and they can all be prepared by conventional preparation methods in the art.

In some embodiments, the product further comprises one or more of an anti-bacterial agent, an anti-caries agent, an anti-allergic agent, an anti-calculus agent, an anti-inflammatory agent, a whitening agent, and a moisturizing agent.

For example, the ergothioneine may be used together with any one, two, three or four of an anti-bacterial agent, anti-caries agent, anti-allergic agent, anti-calculus agent, anti-inflammatory agent, whitening agent and moisturizing agent, or all the above ingredients in a product having the function of inhibiting cell damage caused by acetaldehyde.

In the present application, there is no limitation on the anti-bacterial agent, which may be a conventional anti-bacterial agent in the art, for example, the anti-bacterial agent may be stannous chloride, tetrahydrocurcumin, or triclosan, etc.

In the present application, there is no limitation on the anti-caries agent, which may be an anti-caries agent commonly used in the art, for example, the anti-caries agent may be calcium phosphate, sodium trimetaphosphate, magnesium glycerophosphate, or calcium lactophosphate, etc.

In the present application, there is no limitation on the anti-allergic agent, which may be an anti-allergic agent commonly used in the art, for example, the anti-allergic agent may be dipotassium glycyrrhizinate, potassium fluoride, or potassium chloride, etc.

In the present application, there is no limitation on the anti-calculus agent, which may be an anti-calculus agent commonly used in the art, for example, the anti-calculus agent may be pyrophosphate, tripolyphosphate, or citrate, etc.

In the present application, there is no limitation on the anti-inflammatory agent, which may be an anti-inflammatory agent commonly used in the art, for example, the anti-inflammatory agent may be metronidazole, tinidazole, or ornidazole, etc.

In the present application, there is no limitation on the whitening agent, which may be a whitening agent commonly used in the art, for example, the whitening agent may be peroxide bleach, papain, or glucose oxidase, etc.

In the present application, there is no limitation on the moisturizing agent, which may be a moisturizing agent commonly used in the art, for example, the moisturizing agent may be glycerin, propylene glycol, sorbitol, xylitol, or hyaluronic acid, etc.

In some embodiments, the product further comprises one or more of a pH regulator, a thickener, and an osmotic pressure regulator. The pH regulator may be an acid, alkali, or inorganic salt, etc. available in the art; the thickener may be hydroxyethyl cellulose, carboxymethyl cellulose and a salt thereof, or xanthan gum, etc; the osmotic pressure regulator may be an inorganic salt available in the art.

The present application provides a method for preventing or inhibiting cell damage caused by acetaldehyde for non-therapeutic purposes, the method comprises administering a product containing ergothioneine or the above-mentioned product to a site of damage, thereby reducing the cell damage caused by acetaldehyde.

In some embodiments, a product containing ergothioneine or the above-mentioned product is applied or sprayed to a site of cell damage in the subject, for example, applied or sprayed to oral cavity of the subject.

Acetaldehyde-induced cell damage mainly involves two aspects: oxidative damage and carcinogenesis. There is currently no effective treatment for oxidative damage and carcinogenesis. The inventors of the present application creatively discovered that ergothioneine can alleviate the damage caused by acetaldehyde, which provides a new basis for treating oxidative damage and carcinogenesis caused by acetaldehyde.

The present application provides a method for preventing or inhibiting cell damage caused by acetaldehyde, the method comprises applying ergothioneine to a site of cell damage caused by acetaldehyde, preferably applying ergothioneine to the site before or after the site is contacted with acetaldehyde. In some embodiments, the site comprises oral cavity, nasal cavity, or gastrointestinal tract. In some embodiments, the ergothioneine is used to prevent or inhibit oxidative damage caused by acetaldehyde or to prevent or inhibit cancer caused by acetaldehyde.

The present application provides use of ergothioneine in preventing or inhibiting cell damage caused by acetaldehyde. In some embodiments, the ergothioneine is used to prevent or inhibit oxidative damage caused by acetaldehyde or to prevent or inhibit cancer caused by acetaldehyde. In some embodiments, the site of cell damage caused by acetaldehyde comprises oral cavity, nasal cavity, or gastrointestinal tract.

### EXAMPLES

The present application provides a general and/or specific description of the materials and test methods used in the experiments. In the following examples, unless otherwise specified, % means wt %, i.e., weight percentage. The reagents or instruments used without indicating the manufacturer are all commercially available conventional reagent products.

### Example 1

Experimental materials: ergothioneine, DMEM medium.
Experimental sample solutions: ergothioneine powder was dissolved in DMEM medium to prepare experimental sample solutions with different final concentrations, and the concentrations are shown in Table 1.
Methods: CA9-22 cells purchased from ATCC were co-cultured with different experimental sample solutions for 1 day, then the experimental sample solutions were removed and MTT was added. After 3 hours, the supernatant was carefully aspirated and dimethyl sulfoxide (DMSO) was added. The absorbance of each well at 550 nm was detected by a microplate reader. The untreated well was used as control, the absorbance of the sample well was defined as T, the absorbance of the untreated well was defined as NT, NT was taken as 100%, and the ratio of T/NT of the sample well was taken as the cell activity. The cell activity results are shown in Table 1.

TABLE 1
Cell activity results

| Samples | Cell activity |
|---|---|
| Control group | 100% |
| Ergothioneine-0.1 μM | 101% |
| Ergothioneine-0.5 μM | 100% |
| Ergothioneine-2.5 μM | 107% |
| Ergothioneine-12.5 μM | 106% |

It can be seen from the above Table 1 that, ergothioneine has no cytotoxicity under the test conditions of this experiment.

### Example 2

Experimental materials: acetaldehyde, DMEM medium.
Experimental sample solutions: 17.7 M acetaldehyde was diluted with DMEM medium to prepare experimental sample solutions with different final concentrations, and the concentrations are shown in Table 2.
Methods: After CA9-22 cells were co-cultured with different experimental sample solutions for 1 day, the experimental sample solutions were removed and MTT was added. After 3 hours, the supernatant was carefully aspirated and dimethyl sulfoxide (DMSO) was added. The absorbance of each well at 550 nm was detected by a microplate reader. The untreated well was used as control, the absorbance of the sample well was defined as T, the absorbance of the untreated well was defined as NT, NT was taken as 100%, and the ratio of T/NT of the sample well was taken as the cell activity. The results are shown in Table 2.

TABLE 2
Cell activity results

| Samples | Cell activity |
|---|---|
| Control group | 100% |
| Acetaldehyde-0.02M | 70% |
| Acetaldehyde-0.01M | 89% |
| Acetaldehyde-0.005M | 93% |
| Acetaldehyde-0.001M | 93% |
| Acetaldehyde-0.0001M | 96% |

It can be seen from the above Table 2 that, different concentrations of acetaldehyde will cause cell damage after treating cells. As the concentration of acetaldehyde increases, the damage becomes more obvious. In order to verify the alleviating effect of ergothioneine on oral damage caused by acetaldehyde, this application uses 0.02M acetaldehyde as the cell damage dose.

### Example 3

Experimental materials: acetaldehyde, ergothioneine and DMEM medium.
Experimental sample solutions: ergothioneine powder and 17.7M acetaldehyde were respectively diluted with DMEM medium, and then the two solutions were mixed in different ratios to prepare experimental sample solutions with different final concentrations, and the concentrations are shown in Table 3.
Methods: After CA9-22 cells were co-cultured with different experimental sample solutions for 1 day, the experimental sample solutions were removed and MTT was added. After 3 hours, the supernatant was carefully aspirated and dimethyl sulfoxide (DMSO) was added. The absorbance of each well at 550 nm was detected by a microplate reader. The untreated wells was used as control, the absorbance of the sample wells was defined as T, the absorbance of the untreated well was defined as NT, NT was taken as 100%, and the ratio of T/NT of the sample well was taken as the cell activity. The cell activity results are shown in Table 3. On the basis of meeting the requirements of significant difference, the greater the cell activity %, the better the effect of inhibiting cell damage caused by acetaldehyde.

TABLE 3
Cell activity results

| | Acetaldehyde | | |
|---|---|---|---|
| | concentration | Ergothioneine | |
| Samples | (M) | concentration (μM) | Cell activity |
| 1 | / | / | 100% |
| 2 | 0.02 | / | 70% |
| 3 | 0.02 | 0.01 | 71% |
| 4 | 0.02 | 0.05 | 76% |
| 5 | 0.02 | 0.1 | 81% |
| 6 | 0.02 | 0.5 | 83% |
| 7 | 0.02 | 2.5 | 83% |
| 8 | 0.02 | 12.5 | 85% |

It can be seen from the above table 3 that, the cell activity of the acetaldehyde-treated group is 70%. When ergothioneine was added, the cell activity of CA9-22 increased. The results show that ergothioneine can reduce the cell damage caused by acetaldehyde.

### Comparative Example 1: Effect of Ergothioneine on Cell Damage Under Induction of Inflammatory Factor

Experimental materials: TNF-α (tumor necrosis factor, purchased from Sigma), ergothioneine and DMEM medium.
Experimental sample solutions: ergothioneine powder and TNF-α were diluted with DMEM medium respectively.
Blank group: DMEM medium;
Control group: DMEM medium containing 10 ng/mL TNF-α;
Experimental group: DMEM medium containing 12.5 μM ergothioneine and 10 ng/mL TNF-α. Methods: CA9-22 cells were co-cultured with different experimental sample solutions for 1 day, then the experimental sample solutions were removed and MTT was added. After 3 hours, the supernatant was carefully aspirated and dimethyl sulfoxide (DMSO) was added. The absorbance of each well at 550 nm was detected by a microplate reader. The relative cell activity of the blank group was defined as 100%, and the control group and experimental group were compared with the blank group accordingly.

TNF-α is a pro-inflammatory cytokine that participates in normal inflammatory and immune responses. It can synergistically regulate the production of other cytokines, cell survival and death so as to coordinate tissue homeostasis.

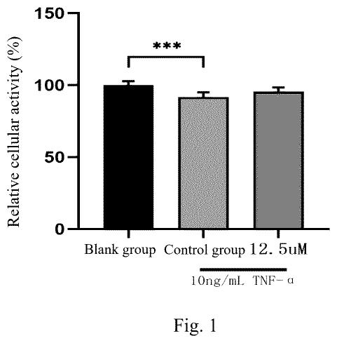

In this experiment, TNF-α was used to stimulate Ca9-22 cells. The experimental results are shown in FIG. 1. Compared the blank group with the control group, it is found that TNF-α significantly affects the cell activity after stimulating Ca9-22 cells with TNF-α, indicating that treating cells with TNF-α will cause cell damage.

In the experimental group, after ergothioneine was added, the decrease of the cell activity of CA9-22 was not significantly improved.

Therefore, based on the above results, it is concluded that ergothioneine has no significant inhibitory effect on the decrease of the cell activity under the induction of TNF-α pro-inflammatory factor, and cannot alleviate the cell damage induced by inflammatory factors.

The above described contents are only preferred embodiments of the present application, and do not constitute any other form of limitation to the present application. Any person skilled in the art may use the technical contents disclosed above to change or modify them into equivalent embodiments with equivalent changes. However, any simple modifications, equivalent changes and modifications made to the above embodiments based on the technical essence of the present application without departing from the content of the technical solution of the present application still fall within the protection scope of the technical solution of the present application.

1. A product for preventing or inhibiting cell damage caused by acetaldehyde, which comprises ergothioneine.

2. The product according to claim 1, wherein the ergothioneine is used to prevent or inhibit oxidative damage caused by acetaldehyde or to prevent or inhibit cancer caused by acetaldehyde.

3. The product according to claim 1, wherein the site of cell damage caused by acetaldehyde comprises oral cavity, nasal cavity or gastrointestinal tract.

4. The product according to claim 1, wherein the product comprises an oral preparation or an external preparation.

5. A method for preventing or inhibiting cell damage caused by acetaldehyde, wherein it comprises administering ergothioneine to a site of cell damage caused by acetaldehyde.

6. The method according to claim 5, wherein it comprises administering the ergothioneine to the site before or after the site is contacted with acetaldehyde.

7. The method according to claim 5, wherein the site comprises oral cavity, nasal cavity or gastrointestinal tract.

8. The method according to claim 5, wherein the ergothioneine is used to prevent or inhibit oxidative damage caused by acetaldehyde or to prevent or inhibit cancer caused by acetaldehyde.

9. Use of ergothioneine in the prevention or inhibition of cell damage caused by acetaldehyde.

10. The use according to claim 9, wherein the ergothioneine is used to prevent or inhibit oxidative damage caused by acetaldehyde or to prevent or inhibit cancer caused by acetaldehyde.

11. The use according to claim 9, wherein the site of cell damage caused by acetaldehyde comprises oral cavity, nasal cavity or gastrointestinal tract.

12. The product according to claim 2, wherein the site of cell damage caused by acetaldehyde comprises oral cavity, nasal cavity or gastrointestinal tract.

13. The product according to claim 2, wherein the product comprises an oral preparation or an external preparation.

14. The product according to claim 3, wherein the product comprises an oral preparation or an external preparation.

15. The method according to claim 6, wherein the site comprises oral cavity, nasal cavity or gastrointestinal tract.

16. The method according to claim 6, wherein the ergothioneine is used to prevent or inhibit oxidative damage caused by acetaldehyde or to prevent or inhibit cancer caused by acetaldehyde.

17. The method according to claim 7, wherein the ergothioneine is used to prevent or inhibit oxidative damage caused by acetaldehyde or to prevent or inhibit cancer caused by acetaldehyde.

18. The use according to claim 10, wherein the site of cell damage caused by acetaldehyde comprises oral cavity, nasal cavity or gastrointestinal tract.
